(19) United States
(12) Patent Application Publication
(10) Pub. No.: US 2024/0206826 A1
(43) Pub. Date: Jun. 27, 2024

(54) BIOSENSOR

(71) Applicant: Murata Manufacturing Co., Ltd., Nagaokakyo-shi (JP)

(72) Inventor: Toru SHIMUTA, Nagaokakyo-shi (JP)

(21) Appl. No.: 18/535,336

(22) Filed: Dec. 11, 2023

Related U.S. Application Data
(63) Continuation of application No. PCT/JP2022/016626, filed on Mar. 31, 2022.

(30) Foreign Application Priority Data
Jun. 29, 2021 (JP) 2021-108194

(51) Int. Cl. A61B 5/00; A61B 5/01; A61B 5/0205; A61B 5/021; A61B 5/024; A61B 5/145; A61B 5/1455

(52) U.S. Cl. CPC A61B 5/7285; A61B 5/02055; A61B 5/02125; A61B 5/6824; A61B 5/6826; A61B 5/7221; A61B 5/01; A61B 5/02427; A61B 5/02438; A61B 5/14532; A61B 5/14552; A61B 2560/0462; A61B 2562/0219; A61B 2562/0271

(57) ABSTRACT

An annular biosensor is provided that includes a main body having in an annular shape to be worn on a finger of a hand or a wrist, a sensor unit for detecting biodata including a blood pressure, an acceleration sensor for detecting an acceleration of the main body and an inclination of the main body relative to a vertical direction, and a control unit that determines, from the detected acceleration, whether a user is sleeping, that estimates a posture of the user during measurement from the detected inclination and determines whether a difference in height between the main body and a heart of the user is within a predetermined range. Based on a result of determining whether the user is sleeping and a result of determining the posture of the user during the measurement, the control unit executes processing of the biodata including the blood pressure.

### CROSS REFERENCE TO RELATED APPLICATIONS

This application is a continuation of International Application No. PCT/JP2022/016626, filed Mar. 31, 2022, which claims priority to Japanese Patent Application No. 2021-108194, filed Jun. 29, 2021, the entire contents of each of which are hereby incorporated by reference in their entirety.

### TECHNICAL FIELD

The present invention relates to a biosensor and, more particularly, to a biosensor for obtaining biodata including a blood pressure, of which a measured value is affected by a difference in height between a measurement location and a heart.

### BACKGROUND

When a location at which a blood pressure is to be measured is positioned at a level higher than a heart, a measured value of the blood pressure decreases corresponding to a difference in hydrostatic pressure inside blood vessels, the difference being caused by gravity. To the contrary, when the location at which the blood pressure is to be measured is positioned at a level lower than the heart, the measured value of the blood pressure increases corresponding to a difference in hydrostatic pressure inside the blood vessels. In more detail, when the location at which the blood pressure is to be measured goes up or down from the height of the heart by 1 cm, the blood pressure (the measured value) changes about 0.7 mmHg.

Japanese Unexamined Patent Application Publication No. 2020-18558 (hereinafter “Patent Document 1”) discloses a blood pressure measuring device that can accurately measure a blood pressure even when a user is in any posture. In more detail, the disclosed blood pressure measuring device includes, in addition to a blood pressure sensor for detecting the blood pressure of the user, one or more sensors to be worn at one or more body locations of the user. Posture information of the user wearing the sensors and height information of the blood pressure sensor are obtained based on sensor information from the one or more sensors. Furthermore, a measured value of the blood pressure measured by the blood pressure sensor is corrected based on the posture information of the user and the height information of the blood pressure sensor.

Patent Document 1 further describes that when a situation that the user is sleeping is determined from a time zone and/or slight variations in acceleration, the blood pressure measuring device can determine in which direction the user orients relative to gravity, because the various sensors are fixedly held on a body of the user.

However, the blood pressure measuring device disclosed in Patent Document 1 requires that, in addition to the blood pressure sensor for measuring the blood pressure, the one or more sensors for obtaining the posture information of the user and the height information of the blood pressure sensor have to be worn at the one or more body locations of the user. Accordingly, handling of the device is intricate and measurement errors caused depending on positions at which the sensors are worn (namely, errors attributable to the handling) tend to generate.

### SUMMARY OF THE INVENTION

Accordingly, it is an object of the present invention to provide a biosensor that obtains, during sleep, biodata including a blood pressure, of which a measured value is affected by a difference in height between a measurement location and a heart (namely, affected by a hydrostatic pressure), that is easy to handle, and that is less likely to cause errors attributable to the handling.

The present invention provides a biosensor including a main body portion formed in an annular shape to be capable of being worn on a finger of a hand or a wrist, a sensor unit disposed in the main body portion and detecting biodata including a blood pressure, an acceleration sensor disposed in the main body portion and detecting an acceleration of the main body portion and an inclination of the main body portion relative to a vertical direction, and a control unit that determines, from the acceleration of the main body portion, whether a user is sleeping, that estimates a posture of the user during measurement from the inclination of the main body portion relative to the vertical direction and determines whether a difference in height between the main body portion and a heart of the user is within a predetermined range, and that, based on a result of determining whether the user is sleeping and a result of determining the posture of the user during the measurement, detects the biodata including the blood pressure with the sensor unit and executes processing of the biodata including the detected blood pressure.

With the biosensor according to the present invention, whether the user is sleeping is determined from the acceleration of the main body portion that is formed in the annular shape to be capable of being worn on the finger of the hand or the wrist, the posture of the user during the measurement is estimated from the inclination of the main body portion relative to the vertical direction, and whether the difference in height between the main body portion and the heart of the user is within the predetermined range is determined. Based on the result of determining whether the user is sleeping and the result of determining the posture of the user during the measurement, the biodata including the blood pressure is detected by the sensor unit, and the processing of the biodata including the detected blood pressure is executed. Therefore, just by attaching the biosensor according to the present invention to be worn on the finger of the hand or the wrist, whether the user is sleeping can be automatically determined, and the biodata including the blood pressure can be processed and obtained in consideration of the posture during the measurement (the posture while sleeping), namely the difference in height between the main body portion and the heart of the user, at that time. The above process eliminates, for example, the necessity of wearing, separately from the blood pressure sensor, one or more sensors for obtaining posture information of the user and height information of the blood pressure sensor at one or more body locations of the user. Hence the biosensor is easier to handle, and errors attributable to the handling are less likely to generate.

According to the exemplary aspects of the present invention, in the biosensor for obtaining the biodata including the blood pressure, of which a measured value is affected by the difference in height between the measurement location and the heart, the biosensor can be more easily handled, and the potential errors attributable to the handling can be reduced.

### BRIEF DESCRIPTION OF DRAWINGS

FIG. 1 illustrates an annular biosensor according to an exemplary embodiment and an overall configuration of a biodata measurement system including the annular biosensor.

FIG. 2 is a block diagram illustrating functional configurations of the annular biosensor according to the exemplary embodiment and the biodata measurement system including the annular biosensor.

FIGS. 3(a) and 3(b) illustrate examples in which the annular biosensor has an asymmetrical shape according to an exemplary embodiment.

FIGS. 4(a) and 4(b) illustrate examples of a hand state in a lying position.

FIGS. 5(a) and 5(b) illustrate additional examples of the hand state in the lying position.

FIG. 6 illustrates an exemplary aspect of an inclination of a center axis of the annular biosensor according to the exemplary embodiment relative to a vertical direction.

FIG. 7 illustrates another exemplary aspect of the inclination of the center axis of the annular biosensor according to the exemplary embodiment relative to the vertical direction.

FIG. 8 is a flowchart illustrating a method of a measurement process for a blood pressure and so on in the annular biosensor according to the exemplary embodiment.

### DETAILED DESCRIPTION OF EMBODIMENTS

An exemplary embodiment of the present invention will be described in detail below with reference to the drawings. It is to be noted that, in the drawings, the same or corresponding components are denoted by the same reference signs. Duplicate description of the same elements denoted by the same reference signs in the drawings is omitted. The following description is made in connection with an example in which the annular biosensor 2 according to the exemplary embodiment is used to form a biodata measurement system 1 together with a portable controller unit 3. The annular biosensor 2 may also be used alone.

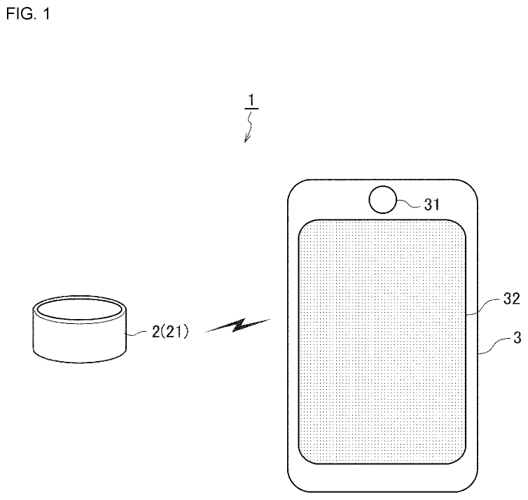

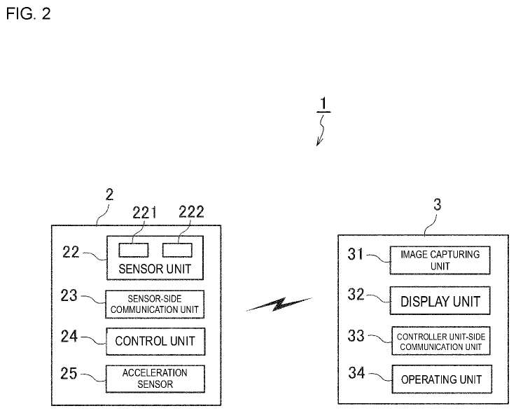

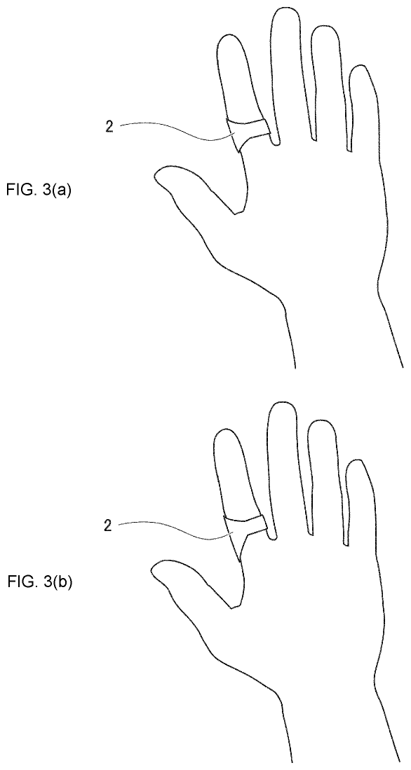

First, the annular biosensor 2 according to the exemplary embodiment and a configuration of the biodata measurement system 1 including the annular biosensor 2 are described with reference to FIGS. 1 to 3 together. FIG. 1 illustrates the annular biosensor 2 and an overall configuration of the biodata measurement system 1 including the annular biosensor 2. FIG. 2 is a block diagram illustrating functional configurations of the annular biosensor 2 and the biodata measurement system 1 including the annular biosensor 2. FIGS. 3(a) and 3(b) illustrate exemplary aspects in which the annular biosensor 2 has an asymmetrical shape. More specifically, FIG. 3(a) represents the case in which the annular biosensor 2 has a left-right asymmetrical shape, and FIG. 3(b) represents the case in which the annular biosensor 2 has an up-down asymmetrical shape.

According to the exemplary aspect, the annular biosensor 2 and the portable controller unit 3 both forming the biodata measurement system 1 are connected to be configured for communicating with each other via wireless communication. In particular, the annular biosensor 2 is a biosensor configured to obtain, during sleep (e.g., while sleeping), biodata including a blood pressure, of which a measured value(s) is affected by a difference in height between a measurement location and a heart (namely, affected by a hydrostatic pressure). The annular biosensor 2 has the features of enabling the biosensor to be more easily handled and making errors attributable to the handling less likely to generate.

As shown, the annular biosensor 2 mainly includes a main body portion 21 (also simply referred to as a main body) that is formed in an annular shape that is configured to be worn on a finger of a hand or a wrist (the former case representing a finger ring type and the latter case representing a wristband type), a sensor unit 22 disposed on the inner surface side of the main body portion 21 and measuring (e.g., detecting) at least a blood pressure, an acceleration sensor 25 disposed in the main body portion 21 and detecting an acceleration of the main body portion 21 (namely, a physical movement of a user) and an inclination of the main body portion 21 relative to a vertical direction, a sensor-side communication unit 23 configured to transmit and receive data (such as measurement data and control data) with respect to the portable controller unit 3, and a control unit 24 configured to determine, from the acceleration of the main body portion 21, whether the user is sleeping, to estimate a posture of the user during measurement from the inclination of the main body portion 21 relative to the vertical direction and to determine whether a difference in height between the main body portion 21 and the heart of the user is within a predetermined range. Based on a result of determining whether the user is sleeping and a result of determining the posture of the user during the measurement, the control unit 24 is configured to detect the biodata including the blood pressure with the sensor unit 22 and to execute processing of the biodata including the detected blood pressure.

According to an exemplary aspect, the control unit 24 is mainly formed by a microprocessor that executes arithmetic operations, an EEPROM that stores programs and so on for causing the microprocessor to execute various kinds of processing, a RAM that temporarily stores data, an input/output interface (I/F), and so on. Various functions of the control unit 24 are realized with execution of the programs stored in the EEPROM, for example, by the microprocessor. In addition, the annular biosensor 2 preferably includes a temperature sensor for detecting a body surface temperature.

The main body portion 21 of the annular biosensor 2 is formed in the annular shape configured to be worn on the finger of the hand (corresponding to the finger ring type). Alternatively, the main body portion 21 is formed in the annular shape that is configured to be worn on the wrist (corresponding to the wristband type). This embodiment is described in connection with an example in which the annular biosensor 2 is a finger ring type biosensor to be worn on the finger of the hand. The annular biosensor 2 is worn on, for example, an index finger of one hand. However, the annular biosensor 2 may be worn on a middle finger, a ring finger, a little finger, or a thumb.

According to an exemplary aspect, the sensor unit 22 is formed as, for example, a photoplethysmographic sensor that includes a light emitting element (e.g., a light emitting portion) 221 and a light receiving element (e.g., a light receiving portion) 222 and that detects a photoplethysmographic signal. The photoplethysmographic sensor optically measures, for example, a pulse by utilizing light absorption characteristics of blood hemoglobin. Hereinafter, the sensor unit 22 is also referred to as the photoplethysmographic sensor 22 in some cases. The sensor unit (e.g., a photoplethysmographic sensor) 22 is disposed on the inner surface side of the main body portion 21.

The sensor unit (e.g., a photoplethysmographic sensor) 22 is preferably arranged in the main body portion 21 such that, when the annular biosensor 2 is worn on one finger of the user, it is located (positioned) on the ventral side of the finger. This is because, in plethysmographic sensors including the photoplethysmographic sensor 22, a biological signal is easier to obtain on the ventral side of a finger than on the dorsal side of the finger.

The sensor unit 22 measures (e.g., detects) at least the blood pressure. This embodiment is described in connection with, for example, a blood pressure sensor that estimates the blood pressure from a photoplethysmographic waveform. In an exemplary aspect, an existing method, such as that described in Japanese Unexamined Patent Application Publication No. 2016-16295 of which the contents are hereby incorporated by reference, can be used as a method of estimating the blood pressure from the photoplethysmographic waveform. In other words, the annular biosensor 2 is a so-called cuffless blood pressure gauge without using a cuff. Other blood pressure estimating techniques (methods), such as a technique utilizing a propagation time of a plethysmographic wave, may also be used.

Even with any type of method being used, however, there is a possibility that the resulting measured value of the blood pressure may become inaccurate due to an influence of the hydrostatic pressure. To avoid the influence of the hydrostatic pressure, measurement of the blood pressure needs to be performed at the height of the heart of the user or nearby. When the measurement of the blood pressure is performed at a level higher than the height of the heart, the measurement result decreases to be lower than a true value, and when the measurement of the blood pressure is performed at a level lower than the height of the heart, the measurement result increases to be higher than the true value. If a difference in height between a position at which the blood pressure is measured and the heart is 10 cm, an error of 7 to 8 mmHg generates in the measured value of the blood pressure. In other words, if the blood pressure is measured at a finger with an arm held in a loosely draped state, a height difference of about 50 cm generates, and an error of 35 to 40 mmHg is caused. When a general user who is not trained unlike health care workers measures the blood pressure, the measurement of the blood pressure is often performed at a level fairly different from the height of the heart of the user, and such measurement causes an error in the measured value of the blood pressure. For that reason, in the method of estimating the blood pressure from the photoplethysmographic wave measured at the finger, it is similarly required to minimize or eliminate the influence of the hydrostatic pressure for the purpose of accurately performing the measurement of the blood pressure.

Furthermore, an existing method, such as that described in Japanese Patent Application No. 2017-506158 of which the contents are hereby incorporated by reference, can be used as a method of estimating a blood sugar level from the photoplethysmographic wave. However, because the photoplethysmographic wave is affected by the blood pressure value at that time, the estimated blood sugar level is also affected. Accordingly, in the case of measurement using a blood sugar level sensor, it is similarly required to hold an appropriate posture during the measurement to restrict the influence of the blood pressure. Moreover, in a posture with an abdomen being in a compressed state, such as a forward leaning posture, the blood pressure may increase. The pulse and/or breathing may further change depending on the posture. From that point of view, the appropriate posture during the measurement needs to be held in not a few cases. The photoplethysmographic waveform includes information of vascular resistance as well. In trying to measure the vascular resistance, the photoplethysmographic waveform is affected by the blood pressure, but variations in the waveform can be reduced by measuring the blood pressure at the height of the heart. While the vascular resistance has been taken as an example, the above description is similarly applied to the case of estimating a blood flow rate, the blood sugar level, and a degree of arteriosclerosis from the waveform. The posture during the measurement further affects a pulse rate, the blood flow rate, the body surface temperature, and the breathing. Thus, variations in the measurement can be reduced by performing the measurement in a predetermined posture. The biodata (e.g., biological information) to be measured may include, for example, a sleeping condition, the plethysmographic wave, the pulse, an oxygen saturation level, the blood sugar level, the body surface temperature, an activity level, the vascular resistance, the blood flow rate, the degree of arteriosclerosis, and the breathing in addition to the blood pressure. Measuring multiple items of the biodata (e.g., information) at the same time as described above makes it possible to estimate whether a physical condition is good, sings of diseases, and so on.

There are various other factors affecting the blood pressure. For example, it is known that meals, drinking, intake of caffeine, and smoking affect the blood pressure. In addition, exercise, waking, work requiring physical movements (such as cleaning), bathing, conversation, mental tension, environments with noise or vibration, and cold environments also affect the blood pressure. The above-mentioned events frequently occur in an awake state, and the timing of the occurrence of the event is difficult to determine. During sleep, influences of the above-mentioned events are reduced, and an appropriate timing to stably measure the blood pressure is given. Thus, by determining from, for example, the activity level, the body surface temperature, and the pulse rate whether the user is sleeping, an awake state and a sleeping state can be distinguished, and accuracy of the measurement can be improved.

Moreover, in the awake state (while awake), the user can take more various kinds of postures than in the sleeping state (while sleeping), and a trunk posture of the user is difficult to estimate from only, for example, the information of an inclination of the hand. By limiting the timing of the measurement to the sleeping state, however, the trunk posture can be more easily estimated from only the information about the hand, and accuracy in estimating the difference in height from the heart can be improved. For example, in a state of a sitting position with a forearm put on an armrest of a chair or in a state in which an arm is put on a backrest of a big sofa, the wrist or the finger is positioned substantially horizontally, and there are no large physical movements. It is hence difficult to distinguish the sitting position and a lying position by using a single sensor. On the other hand, in the sleeping state, because the arm loses its strength and is loosely draped down in many cases, the sitting position and the lying position are easier to distinguish.

In general, the blood pressure is lower in the sleeping state than in the awake state (so-called dipper type). It is also known that, when the blood pressure is substantially equal between the sleeping state and the awake state (so-called non-dipper type), rises in the sleeping state (so-called riser type), or lowers in the sleeping state (so-called extreme-dipper type), the risk of cerebral cardiovascular diseases increases. Measuring the blood pressure during sleep is further advantageous in enabling such a variation type of the nighttime blood pressure to be detected.

In consideration of the above point, the control unit 24 is configured to determine from the acceleration of the main body portion 21 whether the user is sleeping. For example, when the acceleration exceeds above a predetermined value, the control unit 24 determines that the physical movement is detected. Then, when the number of physical movements for a predetermined time is smaller than a predetermined threshold, the control unit 24 determines that the user is sleeping. Although the acceleration may quickly increase due to turning-over or the like even during sleep, a frequency of the turning-over or the like reduces in comparison with that in the awake state. The finger moves at higher frequency in the awake state than, for example, a waist, a chest, and the wrist. Therefore, when an average value of the acceleration for a predetermined time is lower than a predetermined threshold, it may be determined that the user is sleeping. Preferably, by utilizing the fact that a temperature of the finger of the hand rises during sleep, the circadian rhythm is estimated from the body surface temperature at the finger to improve the accuracy in determining the sleeping state in combination with the acceleration. Moreover, because the pulse rate reduces and respiratory variations tend to superimpose on the pulse rate during sleep, the accuracy in determining the sleeping state is improved by additionally measuring a trend of the pulse rate. When the sleeping state is detected, sleeping state information is transmitted.

Physical movements, such as turning-over, occur even during sleep. It is also known that the physical movements increase during REM sleep. The physical movements may also occur due to a sleep disorder. Furthermore, the physical movements may occur due to, for example, a periodic limb movement disorder (PLMD), a restless legs syndrome (RLS), or a sleep apnea syndrome. Upon the occurrence of the physical movements, the blood pressure rises temporarily, and hence the measurement needs to be performed after waiting until the blood pressure is settled. In general, after the lapse of about 5 minutes from the physical movement, the situation can be regarded as being in a resting state. However, after a vigorous physical movement, for example, a longer time is taken until the resting state is reached. In view of the above point, when the acceleration (e.g., physical movement) exceeding above a predetermined value is not detected for a predetermined time or longer, the control unit 24 determines that the situation is in the resting state (or has come into the resting state). The predetermined time (e.g., a determination time) is set to, for example, 5 minutes, but it may be changed depending on the latest detected value of the acceleration (e.g., strength of the physical movement). When it is determined that the situation is in the resting state, resting state information is transmitted. The accuracy of the measurement can be improved by determining the resting state as described above.

Here, execution of each of the sleep determination and the resting state determination takes a time of several minutes or longer. When the physical movements, such as the turning-over, occurs during the above time, the sleep determination is continuously executed in some cases, but the resting state determination is reset. The sleep determination is executed in accordance with, for example, such a criterion that the number of the physical movements occurring with the acceleration exceeding above a first threshold is m or less in 15 minutes or n or less in 90 minutes. On the other hand, the resting state determination is executed in accordance with, for example, such a criterion that the physical movement with the acceleration exceeding above a second threshold does not occur for 5 minutes from the last occurrence. The first threshold and the second threshold are not required to be the same.

The control unit 24 is further configured to determine whether the annular biosensor 2 is worn on the finger of the hand (or the wrist). If posture determination (described in detail later) is executed while the annular biosensor 2 is not worn in place, there is a possibility that the posture may be falsely determined to be appropriate in spite of the posture being not appropriate. The above problem can be avoided by executing the posture determination only when the annular biosensor 2 is worn in place.

Here, a method of determining whether the annular biosensor 2 is worn in place is desirably performed by determining whether the plethysmographic wave is detected by the photoplethysmographic sensor 22. This is because the above determination reduces a possibility of falsely determining that the annular biosensor 2 is worn on the finger in spite of being not worn. However, two or more heartbeats are needed to determine that the plethysmographic wave is detected, and hence the determination may take a time of 3 seconds or longer. In consideration of the above point, the determination may be executed by checking whether the intensity of light received by the photoplethysmographic sensor 22 exceeds below or above a threshold. In the case of the photoplethysmographic sensor 22 being of a reflection type, the intensity of the light received by the sensor reduces if the sensor is not worn in place. Thus, when the intensity of the light received by the sensor exceeds below the threshold, it is regarded that the sensor is not worn in place. In the case of the photoplethysmographic sensor 22 being of a transmission type, the intensity of the light received by the sensor increases if the sensor is not worn in place. Thus, when the intensity of the light received by the sensor exceeds above the threshold, it is regarded that the sensor is not worn in place. With the above-described method, the determination can be executed in a shorter time. However, there is a possibility of determining that the sensor is worn in place (namely, false determination), if something intercepting light is inserted in the annular biosensor 2 regardless of material of that thing. Accordingly, the determination as to whether the annular biosensor 2 is worn on the finger may be executed in combination with, for example, a method of determining that the annular biosensor 2 is not worn in place, if no movements are detected by the acceleration sensor 25, a gyro sensor, or the like, or a method of attaching a temperature sensor to detect the body surface temperature and determining that the annular biosensor 2 is not worn in place, if the detected temperature is a predetermined value or lower.

When it is determined that the annular biosensor 2 is not worn on the finger of the hand or the wrist, the control unit 24 does not execute the determination as to whether the user is sleeping (the sleep determination), the determination as to whether the user is in the resting state (e.g., the resting state determination), and the determination as to the posture of the user during the measurement (e.g., the posture determination).

When it is determined that the user is in the resting state, the control unit 24 estimates the posture of the user during the measurement from the inclination of the annular biosensor 2 (e.g., the main body portion 21) relative to the vertical direction and determines whether the difference in height between the annular biosensor 2 (e.g., the main body portion 21) and the heart of the user is within the predetermined range. Then, based on a result of determining whether the user is sleeping and a result of determining the posture of the user during the measurement, the control unit 24 detects the biodata including the blood pressure with the sensor unit and executes processing of the biodata including the detected blood pressure.

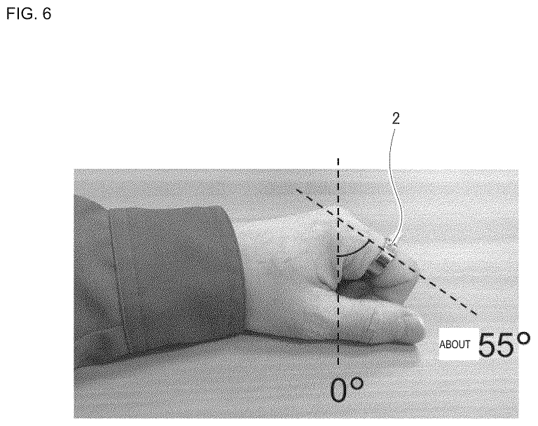

A method of estimating the difference in height between the annular biosensor 2 and the heart according to an exemplary aspect is now described. The principle of the method is similarly applied to an annular biosensor of the wristwatch type or the wristband type which is worn on the wrist. The control unit 24 is configured to estimate the difference in height between the annular biosensor 2 and the heart from the inclination of the center axis of the annular biosensor 2 relative to the vertical direction. In more detail, when the inclination of the center axis of the annular biosensor 2, namely the inclination of an axis of the finger (or the wrist) in a lengthwise direction on which the annular biosensor 2 is worn, relative to the vertical direction is approximately 90°, the user is estimated to be in a lying position (e.g., a supine position, a prone position, or a lateral position) (see FIG. 6). In this case, it is determined that the difference in height between the annular biosensor 2 and the heart is small, and that the measured value can be corrected.

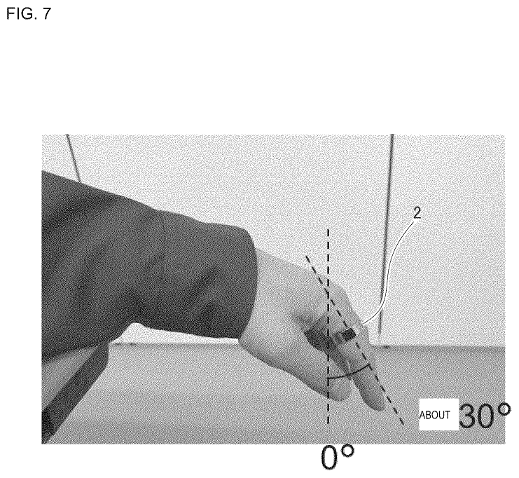

On the other hand, when the inclination of the center axis of the annular biosensor 2, namely the inclination of the lengthwise axis of the finger (or the wrist) on which the annular biosensor 2 is worn, relative to the vertical direction is approximately 0° (when the finger extends in the vertical direction), this indicates that the forearm orients in the vertical direction, and the user is estimated to be in the sitting position or a state in which the arm extends out of a bed and is draped down by gravity (see FIG. 7). In this case, it is determined that the difference in height between the annular biosensor 2 and the heart is large, and that the measured value cannot be corrected. Thus, when the inclination is approximately 0° (when the finger extends in the vertical direction), the user is estimated to be in the posture in which the difference in height from the heart is large, and the measurement of the biodata (such as the blood pressure) that is significantly affected by a deviation in height from the heart is not performed.

The range in which the inclination can be regarded as being appropriately 90° is preferably set to a range of 40 to 90° (see FIG. 6) when the annular biosensor 2 is of the finger ring type and a range of 70 to 90° when the annular biosensor 2 is of the wristwatch type or the wristband type. In the case of using the finger ring type sensor, while the sensor is worn on a proximal phalange, the palm usually curves relative to the wrist, and the proximal phalange also usually curves relative to the palm. Therefore, even with the arm up to the wrist extending at approximately 90°, the proximal phalange may often curve at about 0 to 30° relative to the wrist. For that reason, the above-mentioned range for the finger ring type sensor is set to be relatively wide. The range in which the inclination can be regarded as being appropriately 0° is preferably set to a range of 0 to 40° (see FIG. 7) for the finger ring type sensor and a range of 0 to 70° for the wristwatch or wristband type sensor.

In a state in which the user puts the hand on a floor (e.g., bedclothing) while sleeping, the height of the hand is lower than that of the heart in any of the supine position, the prone position, and the lateral position. Because the heart is positioned nearly at a center of the chest, the height of the hand is lower by a value corresponding to about a half of a chest thickness in the supine position and the prone position. In the lateral position, the height of the hand is lower by a value corresponding to about a half of a chest width. Hence the measured value may be corrected corresponding to the above-described difference in height. According to “AIST Human Body Dimensions Database 1991-1992”, an average value of the chest width (horizontal dimension of the chest) is 288.7 mm, and a half of the average value is 144.4 mm. An average value of the chest thickness (thickness dimension of the chest) is 211.8 mm, and a half of the average value is 105.9 mm. A difference between those two average values is 38.5 mm and corresponds to a deviation of about 3 mmHg in terms of the blood pressure. Such a deviation of the blood pressure is within an allowable range, but the deviation can be reduced by obtaining data of the deviation of the measurement position relative to the height of the heart in many persons as well as acceleration data, and by executing mechanical learning.

In the case of using the annular biosensor 2 of the finger ring type, the height of the measurement position is substantially the same between when the palm faces down and when the palm faces up. A deviation in height substantially corresponding to a thickness of one finger generates depending on whether the measurement location is on the ventral side, the backside, or the lateral side of the finger, or depending on whether the photoplethysmographic sensor is of the reflection type or the transmission type. Such a deviation may be regarded as being allowable or may be corrected to improve the accuracy. In the case of using the annular biosensor of the wristwatch type or the wristband type, when the photoplethysmographic sensor of the reflection type is worn on the backside of the wrist, the height of the sensor elevates by a value corresponding to a thickness of the wrist when the palm faces down in comparison with that when the palm faces up.

The difference in height between the annular biosensor 2 and the heart can be more accurately estimated by using inclinations, relative to the vertical direction, of an axis of the finger (or the wrist) in a widthwise direction and an axis of the finger (or the wrist) in a thickness direction on which the annular biosensor 2 is worn.

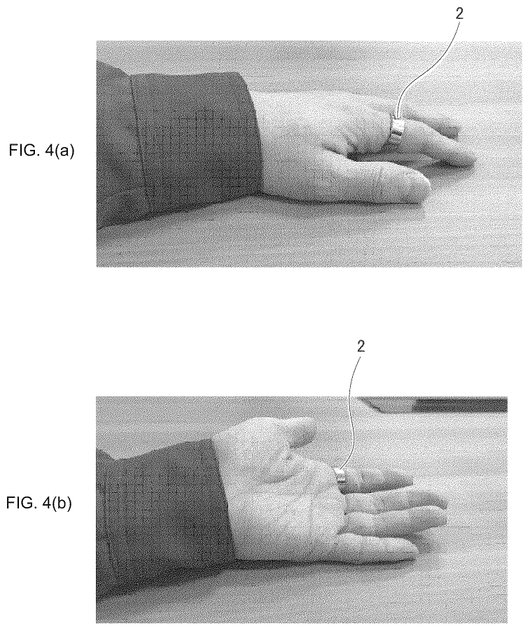

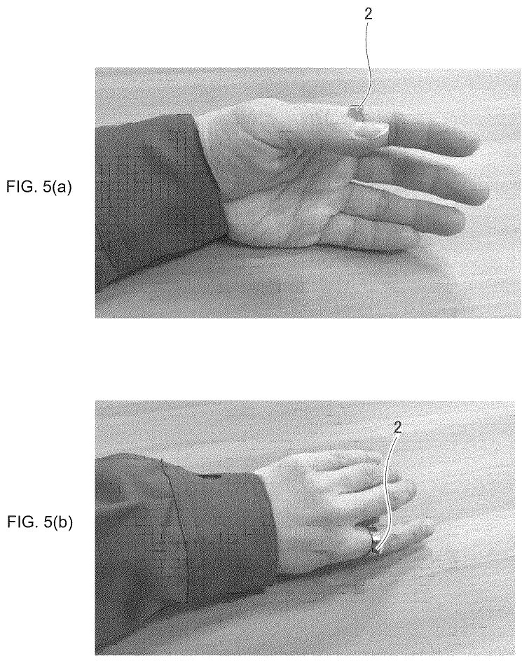

In the case of using the annular biosensor 2 of the finger ring type, the difference in height of the measurement location is not large (such an extent as corresponding to the thickness of one finger) between when the palm faces down and is in touch with the floor (e.g., the bedclothing) (see FIG. 4(a)) and when the back of the hand is in touch with the bedclothing (see FIG. 4(b)). On the other hand, when neither the palm nor the back of the hand is in touch with the bedclothing (namely, when the palm faces substantially in a horizontal direction) (see FIGS. 5(a) and 5(b)), the height of the measurement location elevates in many cases (by a value substantially corresponding to a total thickness of three fingers when the sensor is worn on the index finger). Accordingly, the accuracy in estimating the difference in height from the heart can be improved by estimating the difference in height between the annular biosensor 2 and the heart of the user while an inclination of an axis orthogonal to the center axis of the annular biosensor 2 relative to the vertical direction (for example, an inclination of an X-axis or a Y-axis on an assumption that the center axis is a Z-axis) is further taken into consideration. By using statistical data regarding a width spanning from the index finger (i.e., the second finger) to the little finger (i.e., the fifth finger), the estimation can be made on how much the height of the sensor is elevated when neither the palm nor the back of the hand is in touch with the bedclothing (namely, when the palm faces substantially in the horizontal direction) in comparison with that when the palm faces down and is in touch with the floor (e.g., the bedclothing). For example, it is possible to estimate that the height of the sensor is elevated by a value corresponding to a total width of proximal joints from the index finger (i.e., the second finger) to the ring finger (i.e., the fourth finger) in comparison with that when the palm faces down.

In the case of using the annular biosensor of the wristwatch type or the wristband type, the height of the measurement location is different among when the palm is in touch with the bedclothing, when the back of the hand is in touch with the bedclothing, and when neither the palm nor the back of the hand is in touch with the bedclothing (namely, when the palm faces substantially in the horizontal direction). For example, with the annular biosensor in which the biodata is measured at a center on the backside of the wrist, when the palm is in touch with the bedclothing, the sensor can be estimated to be at a position higher than when the back of the hand is in touch with the bedclothing by a value corresponding to the statistical value regarding the thickness of the wrist. When the palm faces substantially in the horizontal direction, the sensor can be estimated to be at a higher position by a value corresponding to a half of the statistical value regarding the width of the wrist. Thus, the accuracy in estimating the difference in height from the heart can be improved by additionally combining with the inclination of the axis of the finger (or the wrist) in the lengthwise direction relative to the vertical direction.

When the palm faces substantially in the horizontal direction in the case of using the annular biosensor 2 of the finger ring type, there is a possibility that the accuracy in estimating the height of the annular biosensor 2 may deteriorate if both: (1) the finger on which the annular biosensor 2 is worn, and (2) an orientation of the annular biosensor 2 worn on the finger cannot be specified. An estimation result of the height changes depending on whether the annular biosensor 2 is worn on a right hand or a left hand, and on which one of five fingers the annular biosensor 2 is worn. This indicates a possibility that the estimated height may deviate to such an extent as substantially corresponding to three fingers. Accordingly, the control unit 24 estimates the difference in height between the annular biosensor 2 and the heart of the user by obtaining information to specify a position at which the annular biosensor 2 (the main body portion 21) is worn, and by taking the sensor wearing position into consideration. Such a scheme can specify that the annular biosensor 2 is worn on which one of the left and right hands and on which one of the fingers.

In more detail, the above-described scheme can be realized with, for example, a method of prompting the user to input, to the portable controller unit 3, the finger on which the annular biosensor 2 is worn, further prompting the user to move the hand wearing the annular biosensor 2 about an elbow, and specifying an orientation (e.g., frontward or rearward direction) of the annular biosensor 2 from data measured by the acceleration sensor 25 (or the gyro sensor) at that time (the reason is that the orientation (frontward or rearward direction) is not clear even with the determination as to on which one of the fingers the annular biosensor 2 is worn, and hence that the orientation needs to be specified). Another method resides in prompting the user to take an image of the hand wearing the annular biosensor 2 by an image capturing unit 31 (e.g., a camera) in the portable controller unit 3, and specifying the state of the annular biosensor 2 by automatic recognition of the hand and the annular biosensor 2 from the captured image. In the above case, a method of specifying the orientation of the annular biosensor 2 may be realized by determining the orientation from the captured image, or with steps of prompting the user to move the hand wearing the annular biosensor 2 about the elbow as mentioned above and specifying the orientation (e.g., a front or rear direction) of the annular biosensor 2 from the data measured by the acceleration sensor 25 (or the gyro sensor) at that time.

According to an exemplary aspect, the annular biosensor 2 is formed to have such a structure (e.g., a shape) that the annular biosensor 2 cannot be fitted to any finger other than a particular finger. For example, when the main body portion 21 is formed to be asymmetrical with respect to a plane including the center axis or asymmetrical with respect to a plane orthogonal to the center axis (namely, rotationally asymmetrical/left-right asymmetrical in a side view), the annular biosensor 2 can be made difficult to be worn on the finger other than the index finger or the little finger (see FIG. 3(a)). Whether the annular biosensor 2 is to be worn on the index finger or the little finger can be distinguished depending on the diameter of a ring hole. In the case of the annular biosensor 2 having a shape illustrated in FIG. 3(a), because the wider side is positioned on the side closer to the thumb, whether the thumb side is on the upper side or the lower side can be determined by determining, with the acceleration sensor (inclination sensor) 25, whether the above-mentioned wider side faces up or down. In this case, the above estimation can be made similarly regardless of whether the annular biosensor 2 is worn on the left or right hand. Furthermore, the annular biosensor 2 may be formed in a shape allowing the annular biosensor 2 to be worn on only a particular finger by forming the main body portion 21 to be asymmetrical, as illustrated in FIG. 3(b), in the up-down direction (front-rear direction) as well (in an example of FIG. 3(b), the annular biosensor 2 can be worn on only the index finger of the right hand).

As described above, the orientation of the annular biosensor 2 to be worn can be restricted in accordance with whether the annular biosensor 2 is to be worn on the right hand or the left hand, by forming the annular biosensor 2 in an asymmetrical shape between the thumb side and the little finger side. When the palm faces substantially in the horizontal direction, the height of the measurement location changes depending on whether the thumb side is on the lower side or the upper side. However, because the case of the thumb side being on the lower side and the case of the thumb side being on the upper side can be distinguished by restricting the orientation of the annular biosensor 2 to be worn, the accuracy in estimating the difference in height from the heart can be improved.

Moreover, when the annular biosensor 2 is formed with characters or the likes indicated on a lateral surface, the direction of the thumb side can be specified even if the shape of the annular biosensor 2 is left-right symmetrical, by arranging the characters or the likes such that they can be read in the circumferential direction (namely, when they are positioned in a horizontal direction at 90°). The above-mentioned configuration can be realized in the annular biosensor of the wristwatch type or the wristband type by disposing a display or an indicator. When the palm faces substantially in the horizontal direction, the height of the measurement location changes depending on whether the thumb side is on the lower side (FIG. 5(b)) or the upper side (FIG. 5(a)). However, because the case of the thumb side being on the lower side and the case of the thumb side being on the upper side can be distinguished by utilizing the above-described method and restricting the orientation of the annular biosensor 2 to be worn, the accuracy in estimating the difference in height from the heart can be improved.

While the above method is described in connection with an example in which the height of the heart is estimated from the average value of statistical data, the accuracy of the estimation can be improved by using physical information of the user. The physical information of the user may be stored in a memory or a server by prompting the user to input the physical information into the portable controller unit 3 in advance and may be obtained by reading the stored information. Alternatively, the physical information of the user may be obtained by reading data of a health check, for example, the data being stored in a server. Only the height of the user can be given as the physical information, but the physical information more preferably includes the weight and other data (for example, the thickness dimension of the chest) of the user as actually measured values. In fact, the actually measured values of other factors than the height and the weight are not present in many cases. In those cases, values of the other factors are estimated from statistical data. For example, assuming that an average value of the statistical data is denoted by μ, and a standard deviation is denoted by σ, the height of the user is expressed by the following formula (1) using the statistical values μ and σ of the height, and a coefficient a is calculated. The chest thickness dimension of the user can be estimated from the calculated coefficient a and the statistical values of the chest thickness dimension.

Measured value of user=μᵢ+a×σᵢ (1)

Thus, the control unit 24 can be configured to obtain the previously stored physical information of the user and to estimate the height of the heart of the user in consideration of the obtained physical information. With the above-mentioned manner, since the chest thickness and the chest width can be estimated from the height (and the weight), the accuracy in determining whether the annular biosensor 2 is at the height of the heart is improved.

As described above, measuring the blood pressure at the height of the heart in the resting state is important, and the blood pressure value cannot be accurately measured unless the measurement is performed in the appropriate posture. On the other hand, measuring the blood pressure at the height of the heart limits (e.g., restricts) the posture of the user during the measurement, and a difficulty may occur in such a case that continuous data or periodical data is needed. This implies that it is important to calculate reliability of the measured value or to correct the measured value to become substantially equal to the blood pressure value that is to be measured in the appropriate posture during the measurement. As the posture during the measurement deviates from the appropriate posture to a larger extent, inaccuracy in the measured value of the blood pressure increases. Accordingly, by calculating the reliability of the measured value corresponding to the deviation from the appropriate posture, the measured value can be handled in consideration of a risk that the measured value of the blood pressure of the user is deviated from the true value.

From the above point of view, the control unit 24 can be configured to calculate the reliability of the obtained biodata, including the blood pressure, based on a result of determining the posture of the user during the measurement (namely, a result of the posture determination). The calculation of the reliability enables the measured value to be handled in consideration of the risk that the measured value of the blood pressure is deviated from the true value.

More convenience is given to the user by correcting the measured value of the blood pressure to become substantially equal to the blood pressure value that is to be measured in the appropriate posture during the measurement. The control unit 24 may correct the biodata, such as the blood pressure, based on the result of determining the posture of the user during the measurement.

Although the blood pressure value can be corrected if the difference in height between the annular biosensor 2 and the heart can be estimated, the accuracy in estimating the blood pressure is further increased by measuring the blood pressure with the annular biosensor 2 held at the same (vertical) height as the heart. In other words, the accuracy of the blood pressure is more stable when the blood pressure is measured at the same height as the heart every time than when the blood pressure is measured at a position lower or higher than the heart. However, measuring the blood pressure at the same height as the heart implies that the posture of the user during the measurement is restricted. Accordingly, a difficulty may occur in some cases when continuous data or periodical data is needed (namely, there is a possibility of causing pain to the patient). Thus, the continuous data or the periodical data can be obtained by correcting the measured value of the blood pressure to become substantially equal to the blood pressure value that is to be measured in the appropriate posture during the measurement.

When the portable controller unit 3 configured for communicating with the control unit 24 is being operated, the control unit 24 is configured to determine that the user is not sleeping (in the awake state). If the sleep determination is performed from only the acceleration data, a possibility of false determination generates when the physical movements are small in spite of the patient being awake. A probability of the false determination can be reduced by determining the user to be in the awake state when the portable controller unit 3 is being operated. In particular, whether the user is in the sleeping state or the awake state is difficult to determine in a situation of mid-awaking or waking-up. However, the accuracy of the determination can be improved by adding a determination criterion as to whether the portable controller unit 3 is being operated. It is noted that the above method is only applied to the case in which the annular biosensor 2 and the portable controller unit 3 correspond to each other in a one-to-one relation. More specifically, the above method is applied to a system in which only one annular biosensor 2 can be paired with one portable controller unit 3, or a system in which multiple annular biosensors 2 can be each paired with one portable controller unit 3, but which allows only one annular biosensor 2 to be connected to the portable controller unit 3. Stated another way, when the multiple annular biosensors 2 are connectable as in the latter system, there is a possibility that multiple users try to share the portable controller unit 3 at the same time. If it is possible to determine the user operating the portable controller unit 3 from, for example, a login ID of the portable controller unit 3 and to determine that the login user is the same as the user wearing the annular biosensor 2, the above-described method can be applied to even the case in which the multiple annular biosensors 2 are connected.

The sensor-side communication unit 23 is configured to transmit and receive data (such as measurement data and operation/control data) to and from the portable controller unit 3. In this embodiment, Bluetooth® is used as wireless communication standards. In other words, the sensor-side communication unit 23 has transmission and reception functions in accordance with Bluetooth®. It should be appreciated that the wireless communication standards to be used are not limited to Bluetooth®, and other wireless communication standards may also be used. In more detail, the sensor-side communication unit 23 transmits wearing state information of the annular biosensor 2, the sleeping state information, the resting state information, and so on to the portable controller unit 3. In addition, the sensor-side communication unit 23 transmits the obtained biodata, such as the blood pressure, to the portable controller unit 3 at a predetermined timing or period.

On the other hand, the portable controller unit 3 mainly includes the image capturing unit (e.g., a camera) 31 that captures an image (a still image or a moving image), a display unit 32 that is, for example, an LCD display and displays the image captured by the image capturing unit 31, the information, and so on, a controller unit-side communication unit 33 that transmits and receives the data (such as the operation/control data and the measurement data) to and from the annular biosensor 2, and an operating unit 34 that accepts an operation entered by the user. For example, a portable terminal, such as a smartphone, can be preferably used as the portable controller unit 3 that is a control terminal. In this embodiment, the smartphone is used as the portable controller unit 3.

In an example, the portable controller unit 3 is configured to accept an input of information about a wearing location (e.g., a wearing position) of the annular biosensor 2 from the user or takes an image of the wearing location of the annular biosensor 2, specifies the wearing location with, for example, an image analysis, and transmits a result of the image analysis, namely the information about the wearing location of the annular biosensor 2, to the annular biosensor 2.

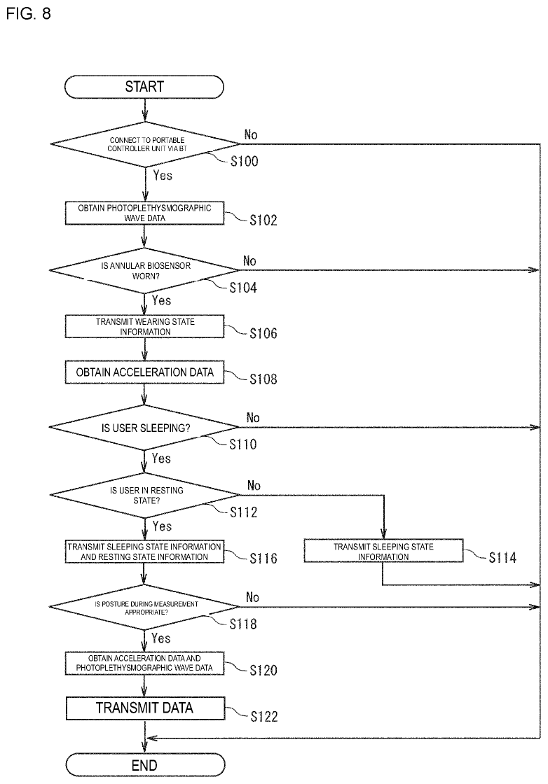

An operation of the annular biosensor 2 will be described below with reference to FIG. 8. FIG. 8 is a flowchart illustrating processing procedures of a measurement process for the blood pressure and so on in the annular biosensor 2. The process illustrated in FIG. 8 is repeatedly executed at a predetermined timing mainly by the annular biosensor 2.

In step S100, whether the annular biosensor 2 is connected to the portable controller unit 3 via Bluetooth® is determined. If the annular biosensor 2 is not connected to the portable controller unit 3, the annular biosensor 2 exits this process once. On the other hand, if the annular biosensor 2 is connected to the portable controller unit 3, the process goes to step S102.

In step S102, the photoplethysmographic signal is obtained. In step S104, whether the annular biosensor 2 is worn on the finger is determined based on the photoplethysmographic signal obtained in step S102. If the annular biosensor 2 is not worn on the finger, the annular biosensor 2 exits this process once. On the other hand, if the annular biosensor 2 is worn on the finger, the process goes to Step S106.

In step S106, the information (e.g., the wearing state information) indicating that the annular biosensor 2 is worn on the finger is transmitted to the portable controller unit 3. In subsequent step S108, the acceleration data (e.g., physical movement data) is obtained.

In step S110, whether the user is sleeping is determined based on the obtained acceleration data (e.g., physical movement data). The method of determining whether the user is sleeping is as per described above, and detailed description of the determination method is omitted here. The above determination does not need to be continuously executed and may be executed at intervals of, for example, 10 minutes. If it is determined that the user is not sleeping (is awake), the annular biosensor 2 exits this process once. On the other hand, if it is determined that the user is sleeping, the process goes to Step S112.

In step S112, whether the user is in the resting state is determined. The method of determining whether the user is in the resting state is as per described above, and detailed description of the determination method is omitted here. If it is determined that the user is not in the resting state, the sleeping state information is transmitted to the portable controller unit 3 in step S114. Then, the annular biosensor 2 exits this process once. On the other hand, if it is determined that the user is in the resting state, the process goes to Step S116.

In step S116, the sleeping state information and the resting state information are transmitted to the portable controller unit 3. Then, in step S118, whether the posture of the user during the measurement is appropriate is determined from the inclination of the main body portion 21 relative to the vertical direction. If the posture of the user during the measurement is not appropriate, the annular biosensor 2 exits this process once. On the other hand, if the posture of the user during the measurement is appropriate, the process goes to Step S120.

In step S120, the photoplethysmographic wave data (e.g., blood pressure data) and the acceleration data (physical movement data) are obtained. Then, in step S122, the photoplethysmographic wave data (e.g., blood pressure data) and the acceleration data (e.g., physical movement data) both obtained in step S120 are transmitted to the portable controller unit 3. After that, the annular biosensor 2 exits this process once.

According to this embodiment, as described in detail above, whether the user is sleeping is determined from the acceleration of the main body portion 21 that is formed in an annular shape to be capable of being worn on the finger of the hand (or the wrist), the posture of the user during the measurement is estimated from the inclination of the main body portion 21 relative to the vertical direction, and whether the difference in height between the annular biosensor 2 (the main body portion 21) and the heart of the user is within the predetermined range is determined. Based on the result of determining whether the user is sleeping and the result of determining the posture of the user during the measurement, the biodata including the blood pressure is detected by the sensor unit 22, and the processing of the biodata including the detected blood pressure is executed. Therefore, just by attaching the annular biosensor 2 to be worn on the finger of the hand (or the wrist), whether the user is sleeping can be automatically determined, and the biodata including the blood pressure can be processed and obtained in consideration of the posture during the measurement (the posture while sleeping), namely of the difference in height between the annular biosensor 2 (the main body portion 21) and the heart of the user, at that time. The above process eliminates, for example, the necessity of wearing, separately from the blood pressure sensor, one or more sensors for obtaining posture information of the user and height information of the blood pressure sensor at one or more body locations of the user. Hence the biosensor is easier to handle, and errors attributable to the handling can be reduced.

As a result, according to this embodiment, in the biosensor for obtaining, during sleep, the biodata including the blood pressure, of which measured value is affected by the difference in height between the measurement location and the heart (namely, affected by the hydrostatic pressure), the biosensor can be handled more easily, and the errors attributable to the handling can be made less likely to generate.

While the blood pressure value may be different depending on the sleeping state or the awake state even with the user being in the same posture, this embodiment can distinguish the sleeping state and the awake state by determining whether the user is in the sleeping state (while sleeping) and can improve the accuracy. Furthermore, in the awake state, the user can take more various kinds of postures than in the sleeping state, and the trunk posture is difficult to estimate from only, for example, the information of the inclination of the hand. By limiting the timing of the measurement to the sleeping state, however, the trunk posture can be more easily estimated from only the information about the hand, and the accuracy in estimating the difference in height from the heart can be improved. In addition, since the difference in height between the annular biosensor 2 and the heart is determined from the inclination of the annular biosensor 2, how far the measured value of the blood pressure is deviated from the true value can be estimated.

While the exemplary embodiment of the present invention has been described above, the present invention is not limited to the above-described embodiment and can be variously modified. For example, while, in the above-described embodiment, the data (e.g., measured data), such as the measured blood pressure, is transmitted to the portable controller unit 3 each time the data is obtained, the measured data may be stored in an EEPROM or a RAM in the annular biosensor 2 and may be read out later (after the measurement). While, in the above-described embodiment, the processing of the biodata, the sleep determination, the resting state determination, and the determination as to the wearing of the biosensor are executed in the control unit 24 of the annular biosensor 2, they may be executed in the portable controller unit 3.

While, in the above-described embodiment, the photoplethysmographic sensor is used as the annular biosensor 2 (the sensor unit 22), the annular biosensor 2 (the sensor unit 22) is not limited to the photoplethysmographic sensor according to alternative aspects.

While, in the above-described embodiment, Bluetooth® is used as the wireless communication standards in accordance with which the data (such as the control data (commands) and the measured data) is transmitted and received between the annular biosensor 2 and the portable controller unit 3, other communication standards, such as BLE (Bluetooth® Low Energy), may also be used instead of Bluetooth®.

### REFERENCE SIGNS LIST

1 biodata measurement system
2 annular biosensor
21 main body portion
22 sensor unit (photoplethysmographic sensor)
221 light emitting element (light emitting portion)
222 light receiving element (light receiving portion)
23 sensor-side communication unit (BT module)
24 control unit
25 acceleration sensor
3 portable controller unit
31 image capturing unit
32 display unit
33 controller unit-side communication unit (BT module)
34 operating unit

1. A biosensor comprising:
a main body having an annular shape that is configured to be worn on a finger of a hand or a wrist of a user;
a sensor disposed in the main body and configured to detect biodata including a blood pressure;
an acceleration sensor disposed in the main body and configured to detect an acceleration of the main body and an inclination of the main body relative to a vertical direction; and
a control unit configured to:
determine, from the acceleration of the main body, whether the user is sleeping,
estimate a posture of the user during a measurement based on the inclination of the main body relative to the vertical direction,
determine whether a difference in height between the main body and a heart of the user is within a predetermined range,
based on a result of determining whether the user is sleeping and a result of determining the posture of the user during the measurement, detect the biodata including the blood pressure with the sensor.

2. The biosensor according to claim 1, wherein the control unit is further configured to execute a processing of the biodata including the detected blood pressure.

3. The biosensor according to claim 1, wherein, when the detected acceleration being greater than or equal to a predetermined value is not detected for at least a predetermined time, the control unit is further configured to determine that the user is in a resting state.

4. The biosensor according to claim 3, wherein, when the user is determined to be in the resting state, the control unit is further configured to estimate the posture of the user during the measurement and determine whether the difference in height between the main body and the heart of the user is within the predetermined range.

5. The biosensor according to claim 4, wherein, when the control unit determines that the biosensor is not worn on the finger of the hand or the wrist, the control unit does not determine (i) whether the user is sleeping, (ii) whether the user is in the resting state, and (iii) the posture of the user during the measurement.

6. The biosensor according to claim 1, wherein the control unit is configured to estimate the difference in height between the biosensor and the heart of the user based on an inclination of a center axis of the biosensor relative to the vertical direction.

7. The biosensor according to claim 6, wherein the control unit is further configured to estimate the difference in height between the biosensor and the heart of the user based on an inclination of an axis orthogonal to the center axis of the biosensor relative to the vertical direction.

8. The biosensor according to claim 1, wherein the main body comprises a shape that is asymmetrical with respect to a plane including a center axis of the main body.

9. The biosensor according to claim 1, wherein the main body comprises a shape that is asymmetrical with respect to a plane orthogonal to the center axis.

10. The biosensor according to claim 1, wherein the control unit is configured to obtain physical information of the user that is stored in advance, and configured to estimate the height of the heart of the user based on the physical information.

11. The biosensor according to claim 1, wherein the biodata includes at least one of a blood sugar level, a pulse, breathing data, a plethysmographic wave, an oxygen saturation level, a body surface temperature, an activity level, and a sleeping condition.

12. The biosensor according to claim 1, wherein the control unit is further configured to calculate reliability of the biodata including the obtained blood pressure based on a result of determining the posture of the user during the measurement.

13. The biosensor according to claim 1, wherein the control unit is configured to correct the biodata including the obtained blood pressure based on a result of determining the posture of the user during the measurement.

14. The biosensor according to claim 1, wherein the control unit is configured to determine that the user is not sleeping when a portable controller unit configured to communicate with the biosensor is being operated.

15. The biosensor according to claim 1, wherein the control unit is configured to obtain information specifying a location where the biosensor is worn and to determine the difference in height between the biosensor and the heart of the user based at least partly on the location where the biosensor is worn.

16. The biosensor according to claim 1, wherein the control unit includes a microprocessor configured to execute a program stored on memory for determining the acceleration of the main body, estimating the posture of the user, determining whether the difference is within the predetermined range, and detecting the biodata.

17. A biosensor comprising:
a main body having an annular shape;
a sensor disposed in the main body and configured to detect blood pressure data of a user;
an acceleration sensor disposed in the main body and configured to detect an acceleration of the main body and an inclination of the main body relative to a vertical direction; and
a control unit configured to:
determine whether the user is sleeping based on the detected acceleration,
estimate a posture of the user during a measurement based on the inclination of the main body relative,
determine whether a difference in height between the main body and a heart of the user is within a predetermined range,
based on a result of determining whether the user is sleeping and a result of determining the posture of the user during the measurement, detect the blood pressure with the sensor.

18. The biosensor according to claim 17, wherein the main body has an annular shape that is configured to be worn on a finger of a hand or a wrist of the user.

19. The biosensor according to claim 17, wherein the control unit is further configured to execute a processing of the detected blood pressure.

20. The biosensor according to claim 17, wherein the control unit is configured to obtain physical information of the user that is stored in advance and configured to estimate the height of the heart of the user based on the physical information.
